(19) United States
(12) Patent Application Publication
(10) Pub. No.: US 2020/0058408 A1
(43) Pub. Date: Feb. 20, 2020

(54) SYSTEMS, METHODS, AND APPARATUS FOR LINKING FAMILY ELECTRONIC MEDICAL RECORDS AND PREDICTION OF MEDICAL CONDITIONS AND HEALTH MANAGEMENT

(71) Applicant: KONINKLIJKE PHILIPS N.V., Eindhoven (NL)

(72) Inventors: Anshul JAIN, Bangalore (IN); Krishnamoorthy PALANISAMY, Bangalore (IN); Nagaraju BUSSA, Bangalore (IN)

(73) Assignee: Koninklijke Philips N.V., Eindhoven (NL)

(21) Appl. No.: 16/342,460

(22) PCT Filed: Oct. 17, 2017
(86) PCT No.: PCT/EP2017/076521

§ 371 Date: Apr. 16, 2019

(30) Foreign Application Priority Data
Oct. 17, 2016 (EP) 16194146.3

(51) Int. Cl. G16H 50/70; G16H 50/20; G16H 10/60; G06N 3/04; G06K 9/62

(52) U.S. Cl. CPC G16H 50/70; G16H 50/20; G06K 9/6297; G06N 3/04; G16H 10/60

(57) ABSTRACT

The embodiments set forth herein relate to systems, methods, and apparatus for using electronic medical records (EMRs) (118, 124, 130, 306, 402, 502) to predict and treat medical conditions. A database (102, 202) of EMR data can be used to compile family trees for different patients. Patterns of medical conditions affecting relatives can be identified and used to predict medical conditions for other relatives. Furthermore, similarities between patients of different family trees (104, 204) can be identified in order to predict and treat medical conditions. In order to accurately predict and treat medical conditions, patient data can be coded to consolidate the amount of patient data available for comparison. Furthermore, coded patient data can be weighted to emphasize data that may be more useful for predicting certain medical conditions.

### TECHNICAL FIELD

The present embodiments are directed generally to improving clinical pathways for disease treatment using hereditary data. More particularly, the embodiments relate to analyzing hereditary data to predict diseases and optimize clinical pathways for treatment, and identify hereditary patterns that are shared by multiple family trees in order to recommend treatments to unrelated patients.

### BACKGROUND

Predicting and diagnosing diseases is, for doctors, a task that involves years of study and practice in order to perform effectively. Recent advances in computer networking have allowed doctors to communicate research and patient outcomes to other doctors who may be working with similar patients. Although the communication of information can provide more resources for doctors to create clinical pathways for treating patients, oftentimes the amount of information can be overwhelming for a doctor to filter through. Additionally, when treatment by a doctor is initially dependent upon a patient first coming to the doctor with an issue, the patient may have already missed opportunities for earlier treatment.

### SUMMARY

The present disclosure is directed to systems, methods, and apparatuses for linking medical records and predicting medical conditions to improve clinical pathways toward treatment. In some embodiments, a method is set forth for providing notifications based on changes to electronic medical records (EMRs). The method can be performed by a computing device and include steps of: receiving event data that identifies a medical event associated with a first patient that is related to a second patient, and receiving measurement data associated with the second patient. The steps can also include identifying a code for classifying the measurement data, wherein the code represents measured medical data that falls within a range of values represented by the code. The steps can further include determining, at least partially based on the code, a probability of the medical event occurring for the second patient, and sending, to a network device associated with the second patient, a notification that relates to the medical event. Additionally, the steps of the method can include receiving relationship data that indicates a familial linkage between the first patient and the second patient. Determining the probability of the medical event occurring for the second patient can be further based on the familial linkage between the first patient and the second patient. Determining the probability of the medical event occurring for the second patient can include determining a first weighted score for the relationship data and a second weighted score for the code that classifies the measurement data. The method can include steps of modifying a first electronic medical record (EMR) associated with the first patient to include an event identifier corresponding to the medical event, and modifying a second electronic EMR associated with the second patient to include the event identifier and the probability of the medical event occurring for the second patient. The steps can further include receiving, from a remote storage device, research data that provides a correspondence between the measurement data and the medical event. Determining the probability of the medical event occurring for the second patient can be further based on the research data.

In other embodiments, a non-transitory computer-readable medium is set forth. The non-transitory computer-readable medium can store instructions that when executed by one or more processors of a computing device, cause the computing device to perform steps that include: identifying first relationship data corresponding to a first group of persons, wherein the first group of persons is identified in electronic medical records (EMR) accessible to the computing device. The steps can also include identifying second relationship data corresponding to a second group of persons, wherein the second group of persons is identified in electronic medical records (EMRs) accessible to the computing device, and wherein the second group of persons are non-relatives of the first group of persons. The steps can further include determining, based on one or more data points associated with the first group of persons and the second group of persons, a similarity measure between the first group of persons and the second group of persons. Additionally, the steps can include, in response to a determination that the similarity measure satisfies one or more criteria, identifying, using the first relationship data and the second relationship data, a common familial linkage between at least two persons in each of the first group of persons and the second group of persons. The steps can further include identifying, in a first electronic medical record (EMR) of the first group of persons, a medical event that is at least partially influenced by the identified common familial linkage, and modifying a second EMR of the second group of persons to include an identifier for the medical event. Identifying a medical event that is at least partially influenced by the identified familial linkage can include determining whether the medical event is associated with a hereditary disease. Additionally, identifying the medical event that is at least partially influenced by the identified familial linkage can include identifying the medical event in at least two EMRs of the first group of persons. In some embodiments, the steps can include modifying a third EMR of the second group of persons to include the identifier for the medical event, wherein the second EMR and the third EMR identify the at least two persons of the second group of persons. The first relationship data can describe at least part of a genealogy of the first group of persons. The common familial linkage is a degree of separation in an ancestral lineage of the at least two persons.

In yet other embodiments, a computing device is set forth as having one or more processors and a memory. The memory can be configured to store instructions that when executed by one or more processors cause the one or more processors to perform steps that include modifying a first electronic medical record (EMR) of a first patient to include treatment data, the treatment data corresponding to a treatment received by a first patient having a condition. The steps can also include identifying, using familial linkage data accessible to the processor, a second EMR of a second patient that is a relative of the first patient, wherein the familial linkage data identifies a first degree of separation in ancestral lineage of the first patient and the second patient. The steps can further include determining, using the familial linkage data, that the second patient has, or is at risk for having, the condition, including: identifying, in a database accessible to the one or more processors, other patients that are non-relatives of the first patient and the second patient and have the condition, and comparing the first degree of separation to a second degree of separation in ancestral lineage of the other patients. The steps can further include modifying a second EMR associated with the second patient to include prospective treatment data that identifies the treatment received by the first patient. Determining that the second patient has, or is at risk for having, the condition can include using the familial linkage data to determine a probability that the second patient has, or will have, the condition. The steps can also include identifying common medical data in the first EMR and the second EMR, and determining, using a data table accessible to the processor, a weight of the common medical data as an indicator of the condition. The common medical data can be a code that is identified in both the first EMR and the second EMR, and the code can represent measured medical data that falls within a range of values represented by the code. The steps can further include generating a report file that identifies the condition and the prospective treatment data, and causing the report file to be transmitted to a personal computing device associated with the second patient. Identifying other patients that are non-relatives of the first patient and the second patient can include analyzing a plurality of family trees stored in the database using a deep neural network. Identifying other patients that are non-relatives of the first patient and the second patient can include analyzing a plurality of family trees stored in the database using a Hidden Markov Model.

A “computing device” can be implemented in numerous ways (e.g., such as with dedicated hardware) to perform various functions discussed herein. A computing device can be a controller that employs one or more microprocessors that may be programmed using software (e.g., microcode) to perform various functions discussed herein. Examples of computing device components that may be employed in various embodiments of the present disclosure include, but are not limited to, conventional microprocessors, application specific integrated circuits (ASICs), and field-programmable gate arrays (FPGAs).

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects of the present invention discussed herein. The terms “program” or “computer program” are used herein in a generic sense to refer to any type of computer code (e.g., software or microcode) that can be employed to program one or more processors or controllers.

In one network implementation, one or more devices coupled to a network may serve as a controller for one or more other devices coupled to the network (e.g., in a master/slave relationship). In another implementation, a networked environment may include one or more dedicated controllers that are configured to control one or more of the devices coupled to the network. Generally, multiple devices coupled to the network each may have access to data that is present on the communications medium or media; however, a given device may be “addressable” in that it is configured to selectively exchange data with (i.e., receive data from and/or transmit data to) the network, based, for example, on one or more particular identifiers (e.g., “addresses”) assigned to it.

The term “network” as used herein refers to any interconnection of two or more devices (including controllers or processors) that facilitates the transport of information (e.g., for device control, data storage, data exchange, etc.) between any two or more devices and/or among multiple devices coupled to the network. As should be readily appreciated, various implementations of networks suitable for interconnecting multiple devices may include any of a variety of network topologies and employ any of a variety of communication protocols. Additionally, in various networks according to the present disclosure, any one connection between two devices may represent a dedicated connection between the two systems, or alternatively a non-dedicated connection. In addition to carrying information intended for the two devices, such a non-dedicated connection may carry information not necessarily intended for either of the two devices (e.g., an open network connection). Furthermore, it should be readily appreciated that various networks of devices as discussed herein may employ one or more wireless, wire/cable, and/or fiber optic links to facilitate information transport throughout the network.

The term “user interface” as used herein refers to an interface between a human user or operator and one or more devices that enables communication between the user and the device(s). Examples of user interfaces that may be employed in various implementations of the present disclosure include, but are not limited to, switches, potentiometers, buttons, dials, sliders, a mouse, keyboard, keypad, various types of game controllers (e.g., joysticks), track balls, display screens, various types of graphical user interfaces (GUIs), touch screens, microphones and other types of sensors that may receive some form of human-generated stimulus and generate a signal in response thereto.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 illustrates a system diagram of an electronic medical record (EMR) system for building a family tree from electronic medical records provided from various sources.

FIG. 2 illustrates an embodiment of an EMR system that provides information about a patient's risks for certain medical conditions based on their relationship to one or more other patients.

FIG. 3 illustrates the EMR system to find all the family trees that are most similar according to certain algorithms.

FIG. 4 illustrates a system for predicting medical conditions of patients by encoding patient input data and ranking similarities between the various encoded patient data.

FIG. 5 illustrates a system for predicting and treating medical conditions of patients by comparing a patient's medical information and family tree to other patients' medical information and family trees.

FIG. 6 illustrates a method for providing medical alerts based on an update provided to a computing device that stores patient medical information, according to some embodiments.

FIG. 7 illustrates a method modifying an EMR based on familial linkage data of different families, according to some embodiments.

FIG. 8 illustrates a method for using familial linkage data to update an EMR to include prospective treatment data, according to some embodiments.

### DETAILED DESCRIPTION

The described embodiments relate to using patient and family tree data to predict, diagnose, and/or treat medical conditions. Typically, medical diagnosis occurs on or after the time of patient testing, therefore it is possible that a medical condition is likely to be present in a patient prior to testing. Oftentimes this can mean harm to the patient may have already occurred as a result of a delayed diagnosis of the medical condition. However, the embodiments herein are set forth to optimize the prediction and treatment of certain medical conditions by employing algorithms for comparing patient records and family trees.

Information about a patient can be stored as medical records in an electronic medical record (EMR) system, which can be a server device or other computing device connected on a network. When multiple patients of the same family have data in the EMR system, the EMR system can link the electronic medical records to form a family tree where the multiple patients and their respective information can be identified. Once a family tree has been created, the EMR system can provide notes and alerts regarding hereditary patterns that can affect a patient. A family tree can be one or more data files that can describe patients' medical histories, relationships (i.e., familial linkage), genetics, lifestyles (e.g., smoker, diet, habits, exercise routine, and/or any other lifestyle descriptors), and/or environments (e.g., location, and/or any other suitable environmental descriptors). When a patient's medical record is updated to include a disease or other condition, the EMR system can calculate a probability that a related person in the same family tree will have the same disease or condition and provide a notification to the related person, doctor, or entity responsible for the related person. Furthermore, the EMR system can access the family trees of other patients and filter the family trees in order to find family trees that are most similar according to certain algorithms. The EMR system can then make predictions about patients in the similar family trees and offer data regarding diagnosis and treatment. Furthermore, if a patient in one family tree is successfully treated according to a particular clinical pathway, recorded in the EMR system, a patient in a similar family tree can be notified of the successful clinical pathway in order that the other patient can take advantage of this for the better outcome.

Predictions regarding the occurrence of a disease for a patient can be based on calculations performed by the EMR system using EMR data related to the patient and individuals in the patients' family. The algorithm for predicting such occurrences can start with identifying a family tree of the patient, which can be stored by the EMR system or by another device that is accessible to the EMR system. If the patient is not associated with a family tree accessible to the EMR system, the EMR system can connect the patient to an existing family tree of a relative or create a new family tree. This will allow the EMR system to track diseases and event occurrences for the patient using data provided by the patient, a patient's relative, a doctor, a medical provider, or any other suitable source of medical data relates to the patient. When the EMR system is updated with this provided data, the data can be linked to the patient's relatives in the family tree, and each patient's relative and/or relative's doctor can receive a notification about the update. Such notifications can be handled by a thread or software running on the EMR system.

The ability of the EMR system to make predictions, diagnosis, and treatment recommendations can be extended outside of a patient's family tree to other family trees using a prediction engine running on the EMR system. The prediction engine can analyze patient data and code certain portions of data based on how they relate to particular diseases. Patient data can be coded according to certain clinical guidelines instead of using raw patient data. Certain metrics of patient data that are measured and fall within a particular range of values can be labeled with a code that is based on the range in which the data falls into. The number of codes can be less than a total number of possible values for the particular metric. For example, a metric for a patient's systolic blood pressure (SBP) can have a range of any positive numbers, and the codes can be “0” for 120 SBP or less, “1” for 120-139 SBP, “2” for 140-159 SBP, “3” for 160-179 SBP, and “4” for 180 or greater SBP. In some embodiments, each code and/or metric can be weighted for indicating the code's and/or metric's importance when predicting a patient's risk for a particular disease. For example, blood pressure can be of greater weight than a patient's height when predicting a diagnosis of cardiovascular disease (CVD). The EMR system can also create a distance mapping for identifying the patients in a family tree that have the most number of similar codes. The distance mapping can not only be used to predict diseases for other relatives in the family tree, but also be compared to distance mappings of other family trees in order to spot similarities that would help the EMR system predict disease occurrences in other families. Furthermore, codes for treatment outcomes can be added to the patient's data in order for the EMR system to also recommend treatments to patients who have not received certain treatments but who are associated with a family tree having a similar distance mapping.

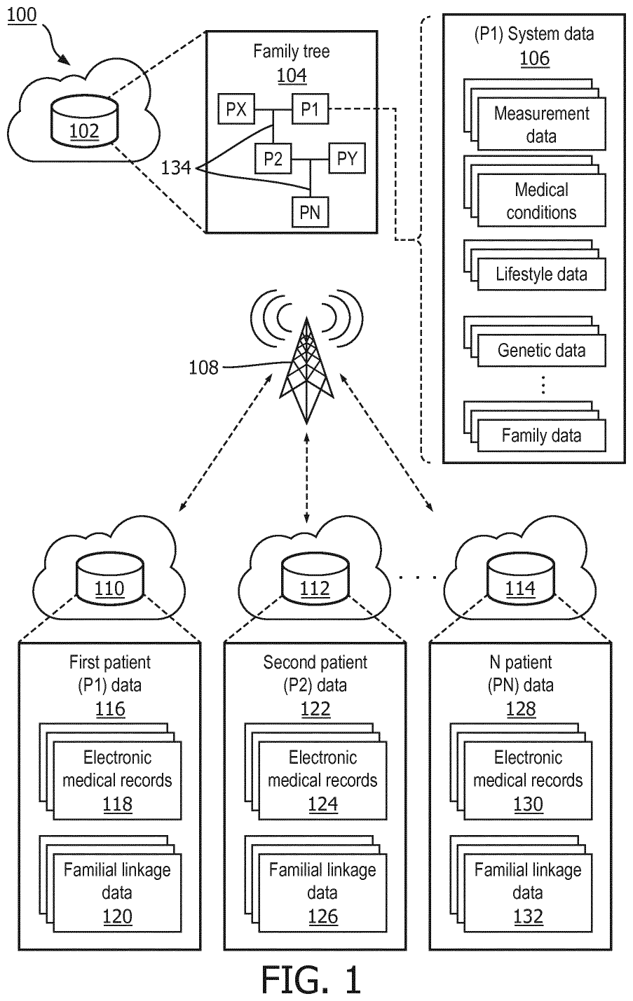

FIG. 1 illustrates a system diagram 100 of an EMR system for building a family tree 104 from electronic medical records (EMRs) provided from various sources. The system diagram 100 includes an EMR database 102 that can collect EMRs from different sources and determine whether different patients (e.g., P1, P2, . . . PN) share ancestral lineage 134. Furthermore, the EMR database 102 can store data representative of a family tree 104 of one or more patients (e.g., P1, P2, . . . PN). The family tree 104 can be derived using family data that can be included in system data stored about a particular patient. For example, the EMR database 102 can store P1 system data 106 corresponding to first patient (P1). P1 system data 106 can include data that relates to at least one of measurement data, medical conditions, lifestyle data, genetic data, and/or family data, as well as any other data suitable for use in predicting medical conditions. Portions of the P1 system data 106 can be used to predict, diagnose, and treat medical conditions of P1, along with any relatives of P1 in the family tree 104. P1 system data 106 can correspond to one or more EMRs received from other databases. The EMR database 102 can connect to other databases over a network device 108, and the network device 108 can connect the EMR database 102 to a private network or public network, such as the internet. Other databases can include a first database 110, a second database 112, and/or any N database 114. Each database can store P1 data 116, P2 data 122, and PN data 128, respectively (wherein N is any positive whole number). Furthermore, the P1 data 116 can include EMRs 118 and familial linkage data 120, the P2 data 122 can include EMRs 124 and familial linkage data 126, and the PN data 128 can include EMRs 130 and familial linkage data 132.

The EMR database 102 can receive one or more of the familial linkage data 120, 126, 132. It should be noted that familial linkage data can be included in the EMRs 118, 124, and 130, respectively, or be separate from the EMRs 118, 124, and 130 in some embodiments. Each familial linkage data 120, 126, and 132 can provide information about the ancestral lineage 134 of each patient P1, P2, and PN, thereby allowing the EMR database 102 to build a family tree 104 from the familial linkage data 120, 126, and 132. In some embodiments ancestral lineage 134 can be derived from third party data, such as an ancestry database that is populated manually by patients. In other embodiments, the ancestral lineage 134 can be derived from the genetic data provided in the EMRs 118, 124, and 130. In yet other embodiments, familial linkage data can be provided by patients or doctors that are aware of the ancestral lineage 134 of patients (e.g., P1 is a parent of P2, and P2 is a parent of PN). By maintaining the family tree 104, the EMR database 102 can predict, diagnose, and help treat diseases that have hereditary dispositions. For example, if EMRs 118 indicate that P1 has a hereditary condition that skips a generation, the EMR database 102 can use this information to provide P3 with information regarding the hereditary condition along with a clinical pathway for treating the condition. Furthermore, if P1 suffers from a disease that is not presently known to be hereditary, the EMR database 102 can identify other family trees where multiple relatives are suffering from the same disease and calculate a risk of P2 or P3 suffering from the same disease. The EMR database 102 can then share the information about the risk of the disease, as well as offer treatment recommendations for the disease. The treatment information can be derived from accessing treatment information about the patients in the other family trees who also had the disease, but who had been treated since their diagnosis.

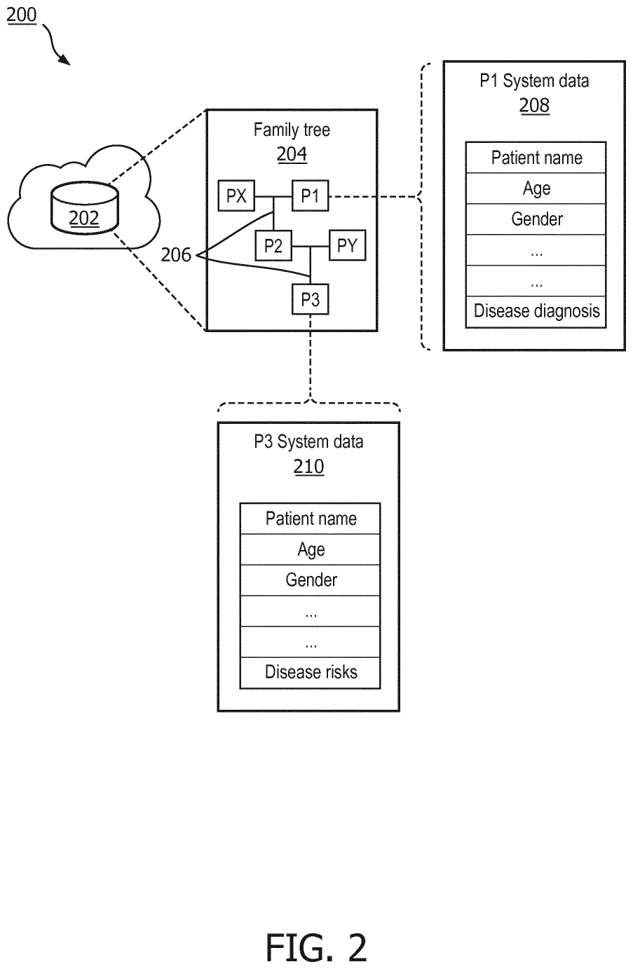

FIG. 2 illustrates an embodiment of an EMR system 200 that provides information about a patient's risks for certain medical conditions based on their relationship to one or more other patients. The EMR system 200 can include an EMR server 202 that stores EMRs for patients that share ancestral lineage 206. Additionally, the EMR server 202 can store a family tree 204 that provides the orders of separation in ancestral lineage 206 between patients. For example, the EMR system 200 can store familial linkage data that indicates P1 is a first degree of separation from P2 and a second degree of separation from P3. Furthermore, the EMR server 202 can store P1 system data 208 and P3 system data 210, which can include medical and non-medical information associated with a first patient (P1) and a third patient (P3), as well as any other number of related or non-related patients. The P1 system data 208 can include data such as, but not limited to, the name of P1, the age of P1, the gender of P1, and a disease diagnosis of P1, and/or any other medical or non-medical related data. When P1 and P3 are linked in the family tree 204 using familial linkage data accessible to the EMR server 202, the P3 system data 210 can be updated to include disease risks. The disease risks can correspond to one or more diseases that are identified in the P1 system data 208 and have been determined to be potential diseases that P3 has now or will have in the future. Risk for diseases or other conditions can be determined using one or more different algorithms for determining patterns or similarities in data. The algorithms can be performed by a predictive engine operating on the EMR server 202.

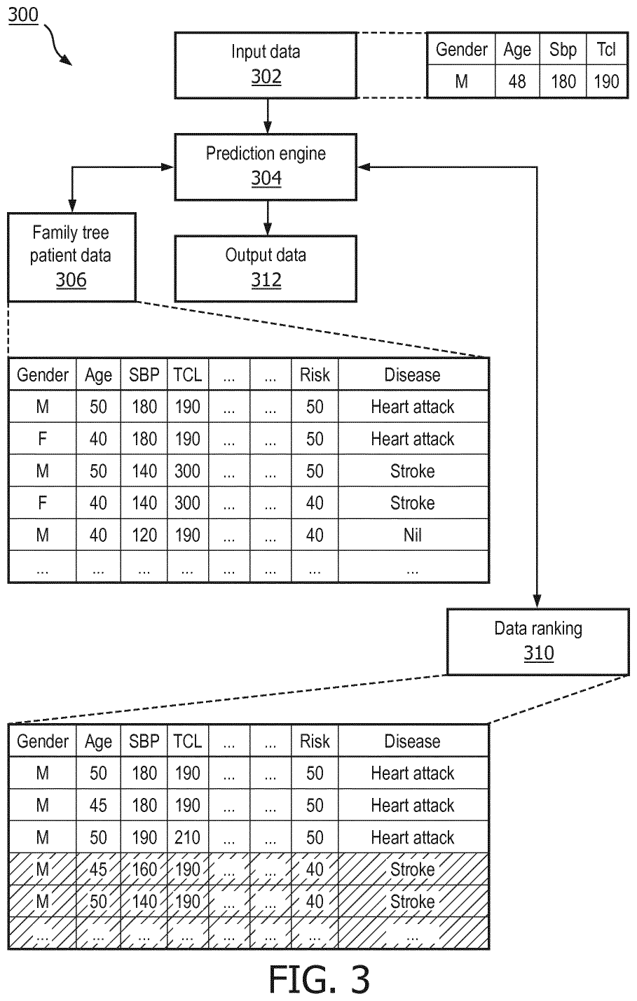

FIG. 3 illustrates a system 300 for predicting medical conditions of patients by ranking family tree patient data according to their similarities to other family trees. The system 300 can include a prediction engine 304, which can be embodied as software operating on an EMR server or EMR database, such as those discussed herein. The prediction engine 304 can receive input data 302 about one or more patients in order to generate predictions about the patients' past, present, and/or future medical conditions. For example, the prediction engine 304 can be used to predict whether a patient is at risk of a medical condition such as a heart attack. The input data 302 for calculating a risk of a heart attack can include basic information such as age and gender, measurements such as systolic blood pressure (SBP) and total cholesterol level (TCL), lifestyle information such as whether the patient smokes, consumes alcohol, or is diabetic, and any prior medical history such as whether the patient has had a previous heart attack, stroke, and/or renal failure. The prediction engine 304 can then access existing family tree patient data 306 to find the most relevant family tree data. A portion of the family tree patient data 306 corresponding to heart attacks can then be ranked according to relevance to the input data 302. A data ranking 310 that ranks the most relevant N entries can then be created by the prediction engine 304. The data ranking 310 can be created by comparing the input data 302 to the family tree patient data 306 and determining a distance or difference between input data 302 and the family tree patient data 306. The family tree patient data 306 that is least different or distant from the input data 302 can be designated as most relevant, and therefore ranked highest in the data ranking 310. A Euclidean distance or weighted Euclidean distance between one or more parameters of the input data 302 and one or more parameters of the patient data 306 can be determined in order to find entries that are most similar. In some embodiments, a coded distance measure can be used to determine a degree of similarity between the input data 302 and the patient data 306.

Using the data ranking 310, relevant information about the patients identified in the data ranking 310 can be used to provide further correlations between the input data 302 and the data ranking 310. Such relevant information can include past medical history and sequence of events that lead to the diagnosis of a heart attack. The prediction engine 304 can use the information about the patients identified in the data ranking 310 to create output data 312 that can be used to predict, diagnose, and/or treat the patient for a disease or condition, such as a heart attack. The output data 312 can include a personalized report that is anonymous as to which patients' data was used to help generate the output data 312. However, the output data 312 can identify information in the EMRs of the patients, which can be relatives or non-relatives of the patient identified in the input data 302.

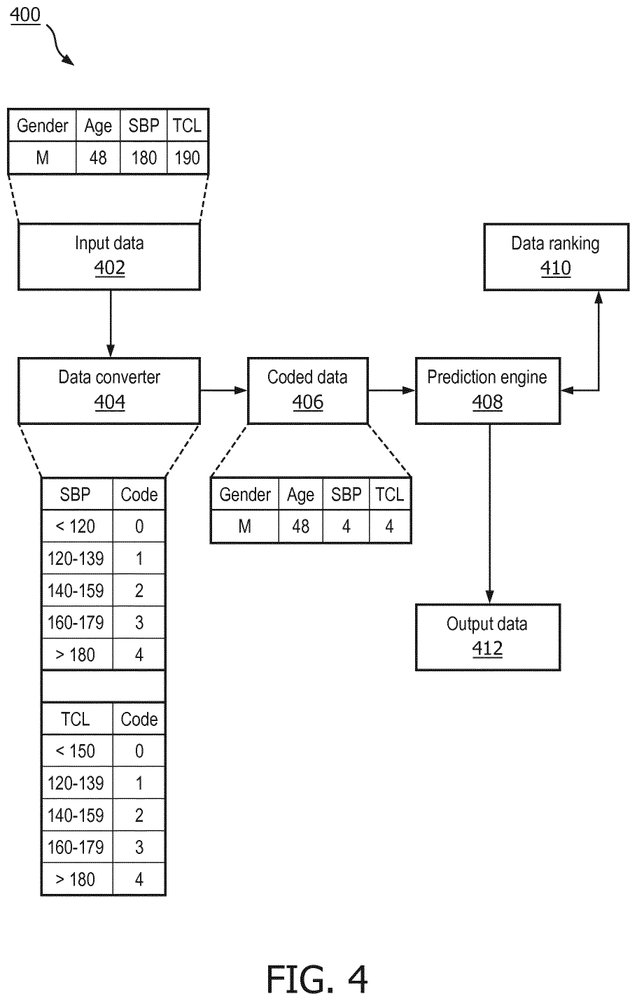

FIG. 4 illustrates a system 400 for predicting medical conditions of patients by encoding patient input data and ranking similarities between the various encoded patient data. The system 400 can include a prediction engine 408, which can be embodied as software operating on an EMR server or EMR database, such as those discussed herein. The prediction engine 408 can use code data to generate predictions about a patient's past, present, and/or future medical conditions. A data converter 404, which can be software that is part of the prediction engine 408 or separate from the prediction engine 408, can convert input data 402 into coded data 406 that can be used to make more accurate predictions about a patient. For example, systolic blood pressure (SBP) can be separated into multiple ranges and each code can be a range label for each of the ranges, as provided in FIG. 4. Codes can be used for any type of patient data and at least two codes can be designated per parameter of patient data. For example, codes for designating someone as a smoker can be 0 for “no” and 1 for “yes.” Furthermore, codes for designating someone who is diabetic can be 0 for “no” and 1 for “yes.” Codes for total cholesterol level (TCL) can be 0 for <150, 1 for 150-189, 2 for 190-229, 3 for 230-269, and 4 for >270. However, it should be noted that any number of ranges can be designated to any number of codes, and that any medical information useful for diagnosing and/or treating a disease can be converted into one or more codes for use by the prediction engine 408.

Once the input data 402 has been converted into coded data 406 by the data converter 404, distance calculations can be performed by the prediction engine 408 using the coded data 406. The coded data 406 can include patient data from an existing patient database and/or data regarding a patient who is new to the system 400. Differences between coded data can be measured in order to find a patient or group of patients with similar codes. For example, for any parameter of medical information, codes can vary from −(M−1) to (M−1), where M is the total number of possible codes for a parameter (e.g., differences of SBPs from FIG. 4 can vary between patients from −4 to 4). Parameters of medical information for two patients can be differenced in order to create a vector, V=[x₁, x₂, . . . , x_{N}], where each value of the vector corresponds to a difference between two coded parameters of two patients' medical information. A total coded distance Dᵢ between two patients can then be calculated as:

Dᵢ=Σₙ₌₁^{N}wₙ(1+|xₙ|)^{|x|} (1)

The value wₙ can be an optional weight that is assigned to one or more parameters of medical information. The weight of a parameter can change based on the disease or condition that the medical information is being used to predict or diagnose. For example, codes for SBP can be weighted greater than the codes for gender of a patient when comparing patient information to determine a risk for heart disease. Furthermore, codes for age can be weighted greater than codes for TCL when comparing patient information to determine a risk for Alzheimer's. The patients having the lowest total coded distance “D” when compared can be considered the most similar patients and ranked accordingly. A probability of a particular event occurring can be calculated as:

$$( 2 ) p = \frac{K}{L}$$

The probability (p) can be equal to the total number of retrieved similar items (K) divided by the number of records in which the disease or event is present (L). In various embodiments, the value of K may be user-selected or selected automatically, e.g., based on one or more pre-defined thresholds. The total number of similar items can change depending on the particular event being considered and/or a threshold set for the coded distance between patients. For example, rarer medical events, or medical events that are less prevalent in a database accessible to the prediction engine 408, can have a higher threshold for the coded distance Dᵢ in order that more output data 412 can be provided for rarer diseases. Additionally, a lower threshold for coded distance Dᵢ can be used for more common events, or medical events that are more prevalent in a database accessible to the prediction engine 408. In this way, for the lower threshold, more codes of patients must be more similar in order for them to be ranked as similar patients for a particular medical event or condition. In some embodiments, treatment data can be included in the input data 402. Treatment data can be designated by at least two codes per treatment. For example, treatment for Alzheimer's can include shunt surgery, and a patient's medical information can indicate whether the patient underwent shunt surgery with a “0” for no, and a “1” for yes. In this way, when distance measurements are taken to find a group of patients that have the most similar medical information, the prediction engine 408 can also identify the patients who have been treated for a particular condition. If a treatment was successful, each of the similar patients can be notified that someone with a similar medical history has been successfully treated for their disease.

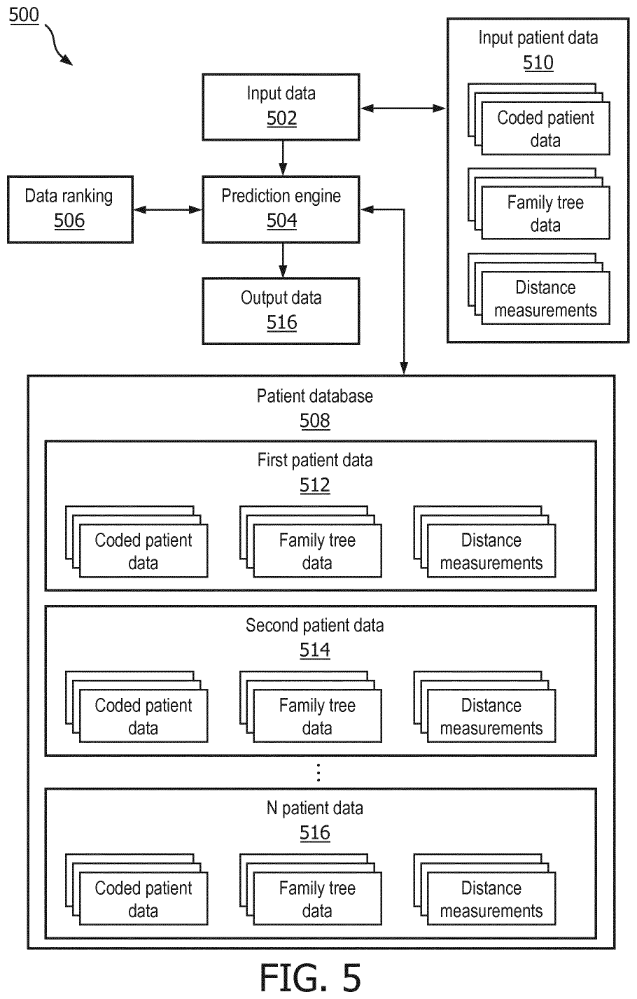

FIG. 5 illustrates a system 500 for predicting and treating medical conditions of patients by comparing a patient's medical information and family tree to other patients' medical information and family trees. The system 500 can include a prediction engine 504, which can be embodied as software operating on an EMR server or EMR database, such as those discussed herein. The prediction engine 504 can receive input data 502 that can include input patient data 510. The input patient data 510 can include coded patient data, family tree data, and/or distance measurements, as discussed herein. The distance measurements can correspond to calculations related to similarities between patient relatives in a family tree. For example, if the coded patient data for a father and a son in the family tree are exactly the same, the distance measurement corresponding to the father and son can be designated as “0.” The prediction engine 504 can be queried to find family tree data from other patients and compare the family tree data to the input patient data 510 provided as input data 502.

Initially, the prediction engine 504 can use the coded patient data of a patient in the input patient data 510 to find similar patients in a patient database 508. Two patients can be considered similar when their distance measurement is within a distance measurement threshold, as determined by the prediction engine 504 or other source. The prediction engine 504 can use the coded patient data of the input data 502 and compare it to the coded patient data of first patient data 512, second patient data 514, and/or N patients' data 516, where N is any real positive whole number. The patients from the patient database 508 can be ranked in a data ranking 506 according to their similarity to the coded patient data of the input data 502. Once a number of patients have been identified and filtered from the patient database 508, their patients' family tree data can be used to determine similarities in the family tree data in the input patient data 510 and the family tree data of the filtered patients. Similarities between patients in a family tree can be determined by using distance measurements between relatives. A patient identified in the data ranking 506 that is most similar to their relatives can be used to help the prediction engine 504 provide output data 516 regarding a patient identified in the input data 502, as well as the patient's relatives. The prediction engine 504 can provide output data 516 for alerting the input patient about medical risks gleaned from analyzing the patient database 508, and alerting relatives of the input patient regarding certain medical risks. When an input patient is different from their relatives according to the distance measurements, the prediction engine 504 can still find patients in the patient database 508 that are most similar to the input patient identified in the input data 502. The identified patients can then be filtered to determine which of the identified patients have relatives that are most similar to the relatives of the input patient. In this way, although the patients and relatives are different, similarities in medical information can be identified between the patients and relatives thereby allowing the prediction engine 504 to help predict, diagnose, and treat diseases of the patients and relatives.

Other techniques may be used to identify similar family trees as well. For example, in some embodiments, prediction engine 504 or another component may employ deep neural networks to analyze patients in the patient database 508 to identify relevant details (e.g., patient codes, etc.) across family trees and identify similar family trees. In other embodiments, Hidden Markov Models (HMM) may be employed to identify similar family trees. For example, a sequence of measurements of a patient may be used as input to the HMM. In yet other embodiments, DNA sequence matching techniques may be employed on DNA sequences associated with members of family trees to identify similar family trees. The complete DNA code in human cells may, in effect, tell the story of a biological family tree. The three most common types of DNA testing work differently by analyzing different parts of DNA code, e.g., Y-DNA, mtDNA and atDNA, any of which can be used to trace a biological family tree. Additionally or alternatively, any combination of DNA sequencing and other aforementioned techniques for identifying similar family trees, such as clinical measurements, may be used to identify similar family trees.

In some embodiments, once a similar family tree is identified, a clinical pathway may be identified using the similar family tree, e.g., using an HMM such as a second order HMM. Suppose that for each identified similar family tree, there are a sequence of clinical measurements, x=x₁, x₂, . . . , x_{N}, and a corresponding clinical pathway sequence, y=y₁, y₂, . . . , y_{N}. For a current family tree, a HMM may be employed to define the following, p(x₁, x₂, . . . , x_{N}, y₁, y₂, . . . , y_{N}), for any such clinical measurements and corresponding clinical pathways of a family tree. In some embodiments, the most likely, or “best,” clinical pathway for x may be determined using the following equations:

$$arg {max}_{{y}_{1} , {y}_{2} , … , {y}_{n}} p ( {x}_{1} , {x}_{2} , … {x}_{n} , {x}_{1} , {y}_{1} , {y}_{2} , … , {y}_{n} )$$

$$p ( {x}_{1} , {x}_{2} , … {x}_{n} , {x}_{1} , {y}_{1} , {y}_{2} , … , {y}_{n + 1 ,} ) = {\prod }_{i = 1}^{n + 1} q ( {y}_{i} \vert {y}_{i - 2 ,} {y}_{i - 1} ) {\prod }_{i = 1}^{n} e ( {x}_{i} \vert {y}_{i} )$$

where q(x|y) and e(x|y) represent, respectively, transition and emission probabilities and may be estimated using various maximum likelihood estimation techniques.

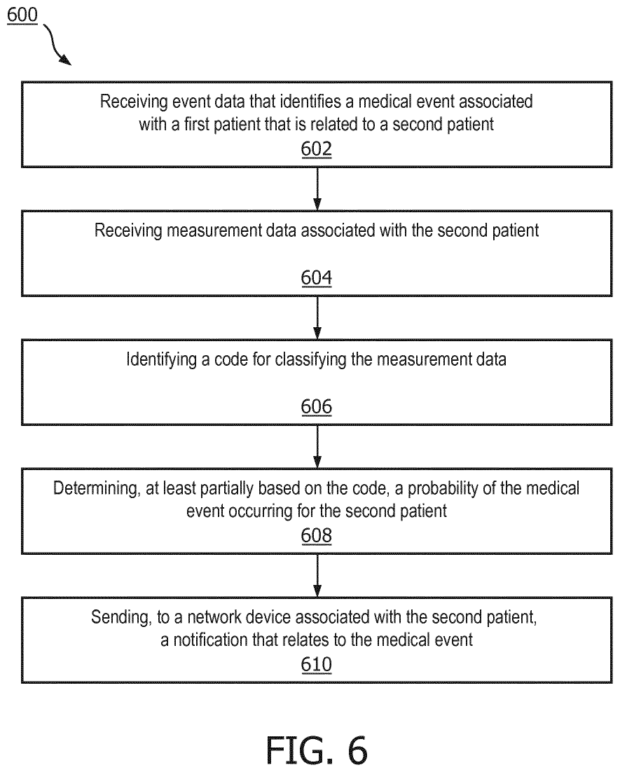

FIG. 6 illustrates a method 600 for providing medical alerts based on an update provided to a computing device that stores patient medical information, according to some embodiments. The method 600 can be performed by any apparatus, computing device, server, and/or database discussed herein. As shown in FIG. 6, the method 600 begins at step 602, and involves the computing device receiving event data that identifies a medical event associated with a first patient that is related to a second patient. The medical event can identify a medical condition, a medical treatment, and/or any other medical information. At step 604, the computing device receives measurement data associated with the second patient. At step 606, the computing device identifies a code for classifying the measurement data. At step 608, the computing device determines, at least partially based on the code, a probability of the medical event occurring for the second patient. The probability can be calculated using a distance measurement between a code of the first patient and the code second patient, as discussed herein. For example, differences between codes associated with each patient can be weighted and summed in order to determine how similar the patients are. This calculation can be performed with a group of related patients who also are associated with the medical event. Using these similarity calculations, a probability of the medical event occurring for the second patient can be determined, as discussed herein. At step 610, the computing device can send, to a network device associated with the second patient, a notification that relates to the medical event. The network device can be a personal computing device of the second patient or a business computing device located at a doctor's office associated with the second patient.

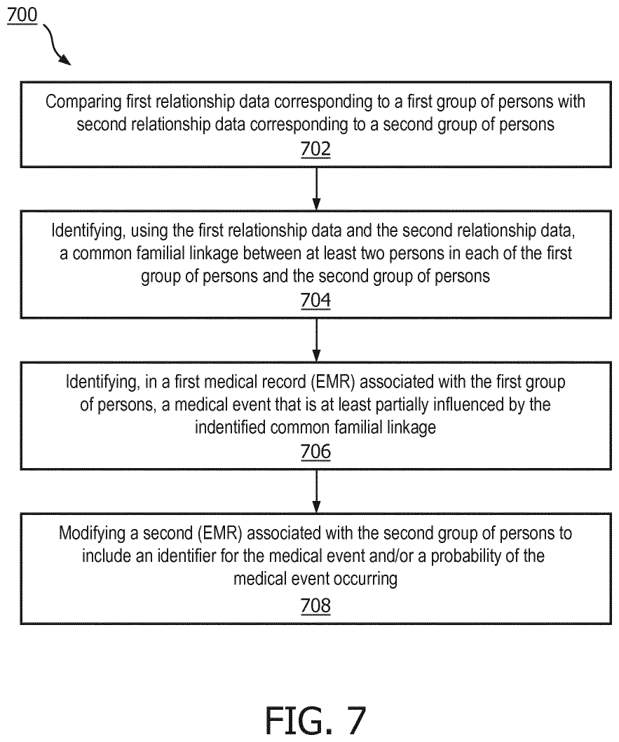

FIG. 7 illustrates a method 700 for modifying an electronic medical record (EMR) based on familial linkage data of different families, according to some embodiments. The method 700 can be performed by any apparatus, computing device, server, and/or database discussed herein. As shown in FIG. 7, the method 700 begins at step 702, and involves a computing device comparing first relationship data corresponding to a first group of persons with second relationship data corresponding to a second group of persons. The first group of persons and the second group of persons can be identified in electronic medical records (EMRs) accessible to the computing device. For example, the computing device can be a database or server that stores the EMRs or is otherwise connected over a network to a device that stores the EMRs. At step 704, the computing device identifies, using the first relationship data and the second relationship data, a common familial linkage between at least two persons in each of the first group of persons and the second group of persons. At step 706, the computing device identifies, in a first medical record (EMR) of the first group of persons, a medical event that is at least partially influenced by the identified common familial linkage. At step 708, the computing device modifies a second EMR of the second group of persons to include an identifier for the medical event. In some embodiments, the identifier can include a name of a medical condition as well as a probability that the medical condition will occur for one or more persons in the second group of persons.

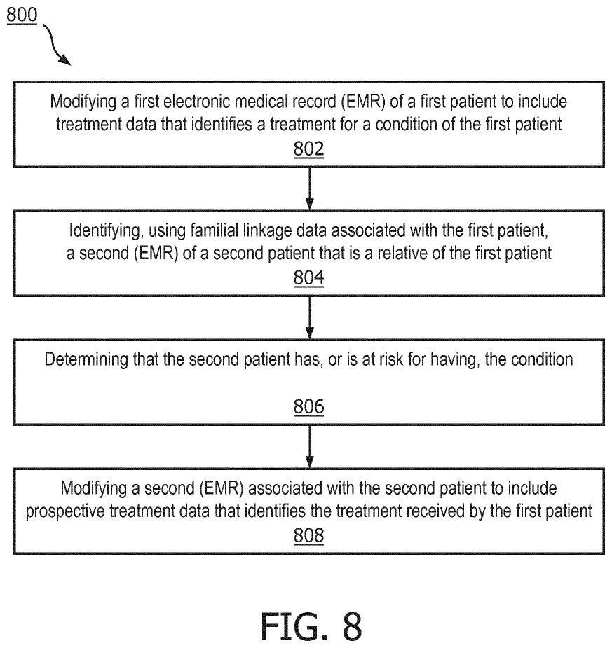

FIG. 8 illustrates a method 800 for using familial linkage data to update an EMR to include prospective treatment data, according to some embodiments. The method 800 can be performed by any apparatus, computing device, server, and/or database discussed herein. As shown in FIG. 8, the method 800 begins at step 802 and involves the computing device modifying a first electronic medical record (EMR) of a first patient to include treatment data. The treatment data can correspond to a treatment received by a first patient having a medical condition. At step 804, the computing device identifies, using familial linkage data associated with the first patient, a second EMR of a second patient that is a relative of the first patient. At step 806, the computing device determines that the second patient has, or is at risk for having, the condition. At step 808, the computing device modifies a second EMR associated with the second patient to include prospective treatment data that identifies the treatment received by the first patient.

Techniques described herein have been described in terms of predicting various ailments and identify treatment plans/preventative measures for existing patients. However, this is not meant to be limiting. In various embodiments, techniques described herein may be used for yet-unborn individuals. For example, in some embodiments, techniques described herein may be employed to automatically identify which vaccines should be provided to a new-born baby given their family tree and similar clinical pathways followed in similar family trees.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be understood that certain expressions and reference signs used in the claims pursuant to Rule 6.2(b) of the Patent Cooperation Treaty (“PCT”) do not limit the scope.

1. A method for providing notifications based on changes to electronic medical records (EMRs), the method comprising steps that include:
by a computing device:
receiving event data that identifies a medical event associated with a first patient that is related to a second patient;
receiving measurement data associated with the second patient;
identifying a code for classifying the measurement data, wherein the code represents measured medical data that falls within a range of values represented by the code;
determining, at least partially based on the code, a probability of the medical event occurring for the second patient; and
sending, to a network device associated with the second patient, a notification that relates to the medical event,
wherein the steps further include:
receiving relationship data that indicates a familial linkage between the first patient and the second patient; and wherein determining the probability of the medical event occurring for the second patient is further based on the familial linkage between the first patient and the second patient.

2. The method of claim 1, wherein determining the probability of the medical event occurring for the second patient includes determining a first weighted score for the relationship data and a second weighted score for the code that classifies the measurement data.

3. The method of claim 1, wherein the steps further include:
modifying a first electronic medical record (EMR) associated with the first patient to include an event identifier corresponding to the medical event; and
modifying a second EMR associated with the second patient to include the event identifier and the probability of the medical event occurring for the second patient.

4. The method of claim 1, wherein the steps further include:
receiving, from a remote storage device, research data that provides a correspondence between the measurement data and the medical event, wherein determining the probability of the medical event occurring for the second patient is further based on the research data.

5. A non-transitory computer-readable medium configured to store instructions that when executed by one or more processors of a computing device, cause the computing device to perform steps that include:
identifying first relationship data corresponding to a first group of persons, wherein the first group of persons is identified in electronic medical records (EMRs) accessible to the computing device;
identifying second relationship data corresponding to a second group of persons, wherein the second group of persons is identified in EMRs accessible to the computing device, and wherein the second group of persons are non-relatives of the first group of persons;
identifying, using the first relationship data and the second relationship data, a common familial linkage between at least two persons in each of the first group of persons and the second group of persons;
identifying, in a first electronic medical record (EMR) of the first group of persons, a medical event that is at least partially influenced by the identified common familial linkage; and
modifying a second EMR of the second group of persons to include an identifier for the medical event.

6. The non-transitory computer-readable medium of claim 5, wherein the step further include:
determining, based on one or more data points associated with the first group of persons and the second group of persons, a similarity measure between the first group of persons and the second group of persons, and
wherein identifying the common familial linkage between at least two persons in each of the first group of persons and the second group of persons is in response to a determination that the similarity measure satisfies one or more criteria.

7. The non-transitory computer-readable medium of claim 5, wherein
identifying the medical event that is at least partially influenced by the identified familial linkage includes determining whether the medical event is associated with a hereditary disease.

8. The non-transitory computer-readable medium of claim 5, wherein
identifying the medical event that is at least partially influenced by the identified familial linkage includes identifying the medical event in at least two EMRs of the first group of persons.

9. The non-transitory computer-readable medium of claim 5, wherein the steps further include:
modifying a third EMR of the second group of persons to include the identifier for the medical event, wherein the second EMR and the third EMR identify the at least two persons of the second group of persons.

10. A computing device, comprising:
one or more processors; and
a memory configured to store instructions that when executed by one or more processors, cause the one or more processors to perform steps that include:
modifying a first electronic medical record (EMR) of a first patient to include treatment data, the treatment data corresponding to a treatment received by a first patient having a condition;
identifying, using familial linkage data accessible to the processor, a second EMR of a second patient that is a relative of the first patient,
determining, using the familial linkage data, that the second patient has, or is at risk for having, the condition.

11. The computing device of claim 10, wherein the familial linkage data identifies a first degree of separation in ancestral lineage of the first patient and the second patient; and wherein determining that the second patient has, or is at risk for having, the condition includes
identifying, in a database accessible to the one or more processors, other patients that are non-relatives of the first patient and the second patient and have the condition, and
comparing the first degree of separation to a second degree of separation in ancestral lineage of the other patients; and
modifying a second EMR associated with the second patient to include prospective treatment data that identifies the treatment received by the first patient.

12. The computing device of claim 10, wherein determining that the second patient has, or is at risk for having, the condition includes using the familial linkage data to determine a probability that the second patient has, or will have, the condition.

13. The computing device of claim 10, wherein the steps further include:
identifying common medical data in the first EMR and the second EMR; and
determining, using a data table accessible to the processor, a weight of the common medical data as an indicator of the condition.

14. The computing device of claim 13, wherein the common medical data is a code that is identified in both the first EMR and the second EMR, and the code represents measured medical data that falls within a range of values represented by the code.

15. The computing device of claim 10, wherein identifying other patients that are non-relatives of the first patient and the second patient includes analyzing a plurality of family trees stored in the database using a deep neural network or Hidden Markov Model.
